(19) United States
(12) Patent Application Publication
(10) Pub. No.: US 2009/0312470 A1
(43) Pub. Date: Dec. 17, 2009

(54) Asymmetric Cyclic Diester Compounds

(75) Inventors: John D. Bradshaw, Solon, OH (US); George F. Schaefer, Strongsville, OH (US); Larry J. Baldwin, Strongsville, OH (US); Ronald J. Raleigh, JR., Mentor, OH (US); Brenda Hollo, Broadview Heights, OH (US)

Correspondence Address: RANKIN, HILL & CLARK LLP, 23755 Lorain Road - Suite 200, North Olmsted, OH, 44070-2224, US

(73) Assignee: FERRO CORPORATION, Cleveland, OH (US)

(21) Appl. No.: 12/421,728

(22) Filed: Apr. 10, 2009

Related U.S. Application Data
(60) Provisional application No. 61/060,520, filed on Jun. 11, 2008.

(51) Int. Cl. C08K 5/12; C07C 69/757; C07D 301/02

(52) U.S. Cl. 524/285; 560/81; 549/518

(57) ABSTRACT

Compounds useful as plasticizers and the synthesis thereof are disclosed. In general, the invention includes mixed alkyl/aryl diesters where the aryl and alkyl ester moieties are attached to a cyclic structure at vicinal carbons. The invention also includes synthetic processes of making such compounds.

### BACKGROUND OF THE INVENTION

1. Field of Invention

The invention relates to plasticizers useful in plasticizing thermoplastic polymers, for example, polyvinyl chloride (PVC). In particular, the invention relates to asymmetric cyclic esters, having aryl and alkyl ester moieties attached to a cyclic structure at vicinal (consecutive) carbons. In many cases, this is carbons in a 1, 2 relationship.

2. Description of Related Art

Plasticizers are compounds or mixtures of compounds that are added to polymer resins to impart softness and flexibility. Phthalic acid diesters, also called phthalates, are the primary plasticizers for most flexible polymer products, especially polymer products formed from polyvinyl chloride (PVC) and other vinyl polymers. Examples of common phthalate plasticizers include: diisononyl phthalate (DINP), butyl benzyl phthalate (BBP), and di-2-ethylhexyl-phthalate (DEHP).

### SUMMARY OF THE INVENTION

The invention relates to mixed alkyl/aryl diester compounds useful in plasticizing polyvinyl chloride (PVC) and other thermoplastic polymers. In particular, the mixed alkyl/aryl diesters of the invention are asymmetric cyclic esters where the aryl and alkyl ester moieties are attached to a cyclic structure at adjacent, or vicinal, carbons. Preferably the cyclic structure is a six-membered ring, such as a cyclohexyl group.

In particular, an embodiment of the invention is a compound including an asymmetric cyclic ester having the formula (I):

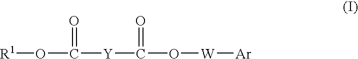

wherein Y is a C₅ to C₈ cyclic alkyl group, wherein R¹ is a straight chain or branched C₂-C₁₈ alkyl group, wherein W is a straight chain or branched C₁-C₅ alkyl group, wherein Ar is a C₆-C₁₅ cyclic aryl group having at least three double bonds, and wherein R¹O(O═)C— and ArWO(O═)C— are attached to vicinal carbons of Y.

A second embodiment of the invention is a process of making an asymmetric cyclic ester comprising: (a) contacting (i) at least one cyclic dicarboxylic acid anhydride with (ii) at least one C₂-C₁₈ aliphatic alcohol, in the presence of (iii) a base to form a reaction mixture and (b) contacting the reaction mixture with a benzyl halide or phenyl-substituted alkyl halide to form a product.

Another embodiment of the invention is a process of making an epoxidized asymmetric cyclic ester comprising: (a) contacting (i) a cyclic dicarboxylic anhydride including one or two unsaturated bonds with (ii) an aliphatic alcohol, in the presence of (iii) a base to form a reaction mixture, (b) contacting the reaction mixture of (a) with a benzyl halide or phenyl-substituted alkyl halide, to form a second reaction mixture, (c) isolating the resulting unsaturated diester and (d) contacting the unsaturated diester with a peracid.

Still another embodiment of the invention is a process of making a saturated asymmetric cyclic ester comprising (a) contacting (i) at least one cyclic dicarboxylic anhydride including one or two unsaturated bonds with (ii) an aliphatic alcohol, in the presence of (iii) a base to form a reaction mixture, (b) contacting the reaction mixture of (a) with a benzyl halide or phenyl-substituted alkyl halide, to form a second reaction mixture, (c) isolating the resulting unsaturated diester, and (d) contacting the unsaturated diester with a hydrogenation catalyst and hydrogen gas.

Yet another embodiment of the invention is a process of making a saturated asymmetric cyclic ester comprising: (a) contacting (i) at least one cyclic dicarboxylic anhydride with (ii) benzyl alcohol or phenyl-substituted alcohol, in the presence of a base to form a reaction mixture, and (b) contacting the reaction mixture of (a) with at least one C₂-C₁₈ alkyl halide to form said mixed diester.

Still another embodiment of the invention is a process of making an unsaturated asymmetric cyclic ester comprising: (a) contacting (i) a maleic anhydride with (ii) an aliphatic alcohol, in the presence of (iii) a base to form a reaction mixture, (b) contacting the reaction mixture of (a) with a benzyl halide or phenyl-substituted alkyl halide, to form an asymmetric maleate diester, and (c) contacting the ester of (b) with a diene to form an unsaturated asymmetric cyclic ester.

Yet another embodiment of the invention is a process of making a saturated asymmetric cyclic ester comprising: (a) contacting (i) a cyclic dicarboxylic anhydride including one, two or three unsaturated bonds with (ii) an aliphatic alcohol, in the presence of (iii) a base to form a reaction mixture including an asymmetric monoester salt, (b) contacting with hydrogen (i) an unsaturated asymmetric monoester acid salt formed in (a) in the presence of (ii) a hydrogenation catalyst, to form a reaction mixture, and (c) contacting the reaction mixture of (b) with a benzyl halide or phenyl-substituted alkyl halide, to form an asymmetric cyclic ester.

Still another embodiment of the invention is a process of making an unsaturated asymmetric cyclic ester comprising: (a) contacting (i) a maleic anhydride with (ii) a benzyl alcohol or phenyl-substituted alcohol, in the presence of (iii) a base to form a reaction mixture, (b) contacting the reaction mixture of (a) with an alkyl halide to form an asymmetric maleate diester, and (c) contacting the ester of (b) with a diene to form an unsaturated asymmetric cyclic ester.

Another embodiment of the invention is a process of making a 1,2-cyclic alkyl/aryl mixed diester comprising (a) contacting at least one (i) cyclic dicarboxylic anhydride with (ii) a C₂-C₁₈ alkyl alcohol, in the presence of (iii) a trialkyl amine to form a reaction mixture and (b) contacting the reaction mixture with a benzyl halide or phenyl-substituted alkyl halide to form a product.

Still another embodiment of the invention is a process of making an epoxidized 1,2-cyclic alkyl/aryl mixed diester comprising: (a) contacting (i) a cyclic compound selected from the group consisting of (1) a cyclic dicarboxylic acid and (2) a cyclic dicarboxylic anhydride and combinations thereof with (i) an alkyl alcohol, in the presence of (ii) a trialkyl amine to form a reaction mixture and (iii) contacting the reaction mixture of (a) with a benzyl halide or phenyl-substituted alkyl halide, to form a second reaction mixture, (b) isolating the resulting unsaturated diester and (c) contacting the unsaturated diester with a peracid.

Yet another embodiment of the invention is a process of making a saturated 1,2-cyclic alkyl/aryl mixed diester comprising: (a) contacting (i) at least one cyclic dicarboxylic anhydride with (ii) an alkyl alcohol, in the presence of (iii) a trialkyl amine to form a reaction mixture (b) contacting the reaction mixture of (a) with a benzyl halide or phenyl-substituted alkyl halide, to form a second reaction mixture, (c) isolating the unsaturated diester, and (d) contacting the unsaturated diester with a hydrogenation catalyst and hydrogen gas.

An embodiment of the invention is a process of making a saturated 1,2-cyclic alkyl/aryl mixed diester comprising: (a) contacting (i) at least one cyclic dicarboxylic anhydride with (ii) benzyl alcohol or phenyl-substituted alcohol, to form a benzyl half-ester, and (b) contacting the benzyl half ester with (i) a C₂-C₁₈ alkyl halide in the presence of (ii) a trialkyl amine to form said mixed diester.

The invention further relates to processes of making plasticized thermoplastic polymers including the addition of any plasticizer herein to a thermoplastic polymer.

The foregoing and other features of the invention are hereinafter more fully described and particularly pointed out in the claims, the following description setting forth in detail certain illustrative embodiments of the invention, these being indicative, however, of but a few of the various ways in which the principles of the invention may be employed.

### DETAILED DESCRIPTION OF THE INVENTION

The compounds of the invention are used to soften thermoplastic polymer resins that would otherwise be brittle and inappropriate for many applications. Plasticizers improve flexibility and tensile strength in such resins.

The present invention relates to asymmetric cyclic ester plasticizers for thermoplastic polymer resins.

In particular, an embodiment of the invention is a compound including an asymmetric cyclic ester having the formula (I):

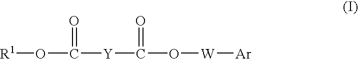

wherein Y is a C₅ to C₈ cyclic alkyl group, wherein R¹ is a straight chain or branched C₂-C₁₈ alkyl group, wherein W is a straight chain or branched C₁-C₅ alkyl group, wherein Ar is a C₆-C₁₅ cyclic aryl group having at least three double bonds, and wherein R¹O(O═)C— and ArWO(O═)C— are attached to vicinal carbons of Y.

A general scheme for synthesizing compounds of this invention includes two critical steps. The first (Reaction 1) is ring opening of the anhydride with an alcohol to form an ester and formation of a salt with the concomitantly formed acid.

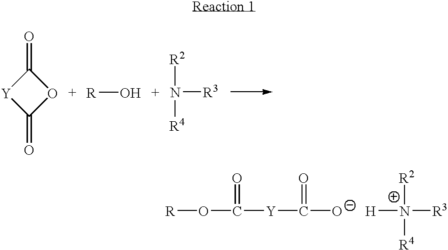

The other critical step (Reaction 2) is the formation of the second ester by reacting the salt with an alkyl halide.

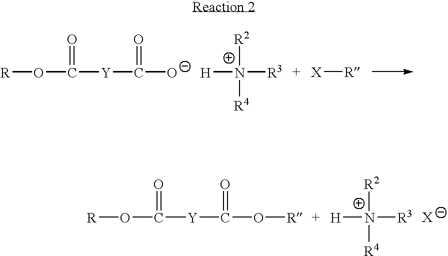

Other intermediate steps may be involved, but the two foregoing reaction steps are common to the general scheme.

The W—Ar group of formula (I) may be derived from either an alcohol in Reaction 1 or from a phenyl-substituted alkyl halide in Reaction 2. Phenyl-substituted alkyl halides [X—W—Ar] that are useful in preparing compounds of this invention include: benzyl halide, 1-halo-1-phenylethane, 1-halo-2-phenylethane, 4-phenyl butyl halide, and others. The corresponding alcohols are useful for introducing the aryl group in Reaction 1.

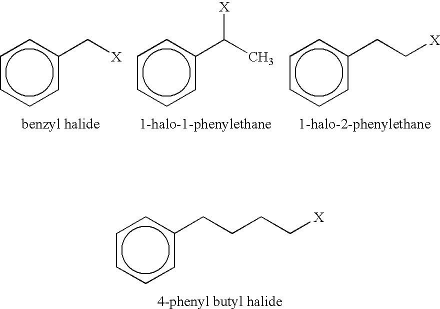

The central ringed group Y may be selected from a variety of 5, 6, 7, or 8 membered rings or bicyclic systems. The group Y may include one or more substituents including, for example, methyl, ethyl, propyl, bridging methylene, bridging ethylene, hydroxyl, bridging oxygen, carboxylic acid, acetyl and others. Preferably, Y is a six-membered ring, based on cyclohexane, which may be unsaturated or substituted, or both (in blends), singly or multiply. Y may also be an epoxidized cyclohexane, a five membered ring such as cyclopentadiene, cyclopentene, or cyclopentane, a seven membered ring such as cycloheptadiene, cycloheptene, or cycloheptane, or an eight membered ring including cyclooctane and the various polyunsaturated forms of cyclooctane.

In particular, the group Y is preferably selected from the group consisting of cyclohexene, cyclohexane, and combinations thereof. The group Y may also be epoxidized cyclohexane, i.e, cyclohexene oxide.

The group Y may be selected from the group consisting of cyclopentadiene; cyclopentene; cyclopentane; cycloheptadiene; cycloheptene; cycloheptane; 3-methyl-4-cyclohexene; 4-methyl-4-cyclohexene; 3-methylcyclohexane; 4-methylcyclohexane; 3,6-epoxy-4-cyclohexene; 3,6-epoxycyclohexane; cis-5-norbornene; norbornene; methyl-5-norbornene; bicyclo[2.2.2]oct-5-ene; bicyclo[2.2.2]octane; and combinations thereof.

Alternatively, the group Y may be a residue of a carboxylic acid anhydride, in particular 4-cyclohexene-1,2-dicarboxylic acid anhydride; cyclohexane-1,2-dicarboxylic acid anhydride; 1,2-dicarboxy-4-alkyl cyclohex-4-ene anhydride; 1,2-dicarboxy-3-alkyl cyclohex-3-ene anhydride; cis-5-norbornene-2,3-dicarboxylic anhydride; norbornane-2,3-dicarboxylic anhydride; methyl-5-norbornene-2,3-dicarboxylic anhydride; 3,6-epoxy-cyclohexene-1,2-dicarboxylic acid anhydride; 3,6-cyclohexane-1,2-dicarboxylic acid anhydride; bicyclo[2.2.2]oct-5-ene-2,3-dicarboxylic anhydride; bicyclo[2.2.2]octane-2,3-dicarboxylic anhydride; and combinations thereof.

The aliphatic alcohols (which donate the R¹ group of the inventive diesters) used in forming the asymmetric cyclic esters of the invention can (but need not) be halogenated. Such alcohols can be linear, branched, or have cyclic moieties. Preferably, the aliphatic alcohols contain 2 to about 18 carbon atoms, more preferably 4 to 10 carbon atoms, and most preferably 5 to 9 carbon atoms. Alternatively, the alcohols may include 2 to 8 carbons. Suitable aliphatic alcohols include, for example, ethanol, bromoethanol, n-propanol, isopropanol, 2-chloropropanol, 3-chloropropanol, 2-methylpropanol, 2-ethylpropanol, n-butanol, isobutanol, tert-butanol, 2-methylbutanol, 3-methylbutanol, 2-ethylbutanol, 2,2-dimethylbutanol, 2,3-dimethylbutanol, 3,3-dimethylbutanol, 2-methylpentanol, 3-methylpentanol, 4-methylpentanol, 2-ethylpentanol, 3 ethylpentanol, 4-ethylpentanol, cyclopentyl ethanol, cyclopentyl propanol, cyclopentyl hexanol, cyclopentyl butanol, cyclopentyl pentanol, cyclohexanol, cyclohexyl ethanol, 2-ethylhexanol, n-nonanol, isononanol, tert-nonanol, decanol, undecanol, propylheptanol, dodecanol, oleyl alcohol and stearyl alcohol. The various forms of nonanol and decanol are preferred, and isononanol and 2-propylheptanol are most preferred. Other aliphatic alcohols not named herein and other forms of alkyl alcohols named herein are also suitable, provided they have no more than 18 carbons.

Preferably, R¹ includes 2 to about 18 carbon atoms, more preferably 4 to 10 carbon atoms, for example C₄ to C₁₀ straight chain or branched alkyl groups, and more preferably 5 to 9 carbon atoms. A blend of compounds having different embodiments of general Formula I, having any of the Y, R¹, W, and Ar groups disclosed herein is envisioned, in any combination.

The W group includes 1 to 5 carbon atoms, preferably 1 or 2 carbon atoms, and more preferably one carbon atom. The W group is provided by alkyl groups attached to the Ar group, in the reactant Ar—W—X, which participates in the esterification reactions disclosed herein. The formula Ar—W—X can stand for a benzyl halide, an alkyl substituted benzyl halide, or a phenyl substituted alkyl halide.

The aryl reactants (which donate the Ar group of the inventive diesters) used in the esterification reaction mixture have one or more aromatic rings. Various substituents, including alkyl groups, may be present on the rings. Accordingly, the group Ar may be selected from the group consisting of benzene; methylbenzene; dimethyl benzene, ditertiary butyl benzene; napththalene, anthracene, cumene and combinations thereof.

The aryl reactants, which include the W and Ar groups of the diesters of the invention, are preferably halogenated with at least one of F, Cl, Br, and I, more preferably Br or Cl, and most preferably Cl. Suitable halogenated aryl reactants include, for example, benzyl halide, 2-methylbenzyl halide, 3-methylbenzyl halide, 4-methylbenzyl halide, 2-ethylbenzyl halide, 3-ethylbenzyl halide, 4-ethylbenzyl halide, 4-isopropylbenzyl halide, 4-tertiary butylbenzyl halide, 4-(1-methylpropyl)benzyl halide, 2-ethoxybenzyl halide, 3,4-dimethoxybenzyl halide, 4-methoxy-2-methylbenzyl halide, 4-acetylbenzyl halide, acetylmandelyl chloride, 2-chlorobenzyl halide, 3-chlorobenzyl halide, 4-chlorobenzyl halide, 2,3-dibromobenzyl halide, 2,4-di-iodobenzyl halide, 2,6-dibrombenzyl halide, 3,4-dibromobenzyl halide, 3,5-dichlorobenzyl halide, 2,3,5-tribromobenzyl halide, 2,4,6-trichlorobenzyl halide, 2,3,4,5,6-pentabromobenzyl halide, 1-phenyl ethyl halide, 2-phenyl ethyl halide, 1-phenyl butyl halide, 4-phenyl butyl halide, 1-naphthyl halide, and 2-naphthyl halide. Preferred are the chloride forms of the above halides. Most preferred is benzyl chloride.

Asymmetric cyclic ester compounds of the invention include benzyl isononyl cyclohex-4-ene-1,2-dicarboxylate; benzyl isononyl cyclohexane-1,2-dicarboxylate; butyl benzyl 4-cyclohexene-1,2-carboxylate; butyl benzyl cyclohexane-1,2-dicarboxylate; benzyl 2-propylheptyl 4-cyclohexene-1,2-dicarboxylate; benzyl 2-propylheptyl cyclohexane-1,2-dicarboxylate; benzyl butyl 3-methylcyclohexane-1,2-dicarboxylate; benzyl isononyl 3-methylcyclohexane-1,2-dicarboxylate; benzyl 2-propylheptyl 3-methylcyclohexane-1,2-dicarboxylate; benzyl butyl 4-methylcyclohexane-1,2-dicarboxylate; benzyl isononyl 4-methylcyclohexane-1,2-dicarboxylate; benzyl 2-propylheptyl 4-methylcyclohexane-1,2-dicarboxylate; benzyl butyl 3-methylcyclohex-4-ene-1,2-dicarboxylate; benzyl isononyl 3-methylcyclohex-4-ene-1,2-dicarboxylate; benzyl 2-propylheptyl 3-methylcyclohex-4-ene-1,2-dicarboxylate; benzyl butyl 4-methylcyclohex-4-ene-1,2-dicarboxylate; benzyl isononyl 4-methylcyclohex-4-ene-1,2-dicarboxylate; benzyl 2-propylheptyl 4-methylcyclohex-4-ene-1,2-dicarboxylate; benzyl butyl norbornane-2,3-dicarboxylate; benzyl isononyl norbornane-2,3-dicarboxylate; benzyl 2-propylheptyl norbornane-2,3-dicarboxylate; benzyl butyl 5-norbornene-2,3-dicarboxylate; benzyl isononyl 5-norbornene-2,3-dicarboxylate; benzyl 2-propylheptyl 5-norbornene-2,3-dicarboxylate; benzyl butyl methylnorbornane-2,3-dicarboxylate; benzyl isononyl methylnorbornane-2,3-dicarboxylate; benzyl 2-propylheptyl methylnorbornane-2,3-dicarboxylate; benzyl butyl 1-methyl-5-norbornene-2,3-dicarboxylate; benzyl isononyl 1-methyl-5-norbornene-2,3-dicarboxylate; benzyl 2-propylheptyl 1-methyl-5-norbornene-2,3-dicarboxylate; benzyl butyl 3,6-epoxy-cyclohexane-1,2-dicarboxylate; benzyl isononyl 3,6-epoxy-cyclohexane-1,2-dicarboxylate; benzyl 2-propylheptyl 3,6-epoxy-cyclohexane-1,2-dicarboxylate; benzyl butyl 3,6-epoxy-cyclohexene-1,2-dicarboxylate; benzyl isononyl 3,6-epoxy-cyclohexene-1,2-dicarboxylate; benzyl 2-propylheptyl 3,6-epoxy-cyclohexene-1,2-dicarboxylate; benzyl butyl bicyclo[2.2.2]octane-2,3-dicarboxylate; benzyl isononyl bicyclo[2.2.2]octane-2,3-dicarboxylate; benzyl 2-propylheptyl bicyclo[2.2.2]octane-2,3-dicarboxylate; benzyl butyl bicyclo[2.2.2]oct-5-ene-2,3-dicarboxylate; benzyl isononyl bicyclo[2.2.2]oct-5-ene-2,3-dicarboxylate; benzyl 2-propylheptyl bicyclo[2.2.2]oct-5-ene-2,3-dicarboxylate; butyl 1-phenylethyl cyclohex-4-ene-1,2-dicarboxylate; butyl 1-phenylethyl cyclohexane-1,2-dicarboxylate; butyl 1-phenylethyl 3-methylcyclohex-4-ene-1,2-dicarboxylate; butyl 1-phenylethyl 3-methylcyclohexane-1,2-dicarboxylate; butyl 1-phenylethyl 4-methylcyclohex-4-ene-1,2-dicarboxylate; butyl 1-phenylethyl 4-methylcyclohexane-1,2-dicarboxylate; isononyl 1-phenylethyl cyclohex-4-ene-1,2-dicarboxylate; isononyl 1-phenylethyl cyclohexane-1,2-dicarboxylate; isononyl 1-phenylethyl 3-methylcyclohex-4-ene-1,2-dicarboxylate; isononyl 1-phenylethyl 3-methylcyclohexane-1,2-dicarboxylate; isononyl 1-phenylethyl 4-methylcyclohex-4-ene-1,2-dicarboxylate; isononyl 1-phenylethyl 4-methylcyclohexane-1,2-dicarboxylate; 1-phenylethyl 2-propylheptyl cyclohex-4-ene-1,2-dicarboxylate; 1-phenylethyl 2-propylheptyl cyclohexane-1,2-dicarboxylate; 1-phenylethyl 2-propylheptyl 3-methylcyclohex-4-ene-1,2-dicarboxylate; 1-phenylethyl 2-propylheptyl 3-methylcyclohexane-1,2-dicarboxylate; benzyl butyl 4,5-epoxycyclohexane-1,2-dicarboxylate; benzyl isononyl 4,5-epoxycyclohexane-1,2-dicarboxylate; benzyl 2-propylheptyl 4,5-epoxycyclohexane-1,2-dicarboxylate; benzyl butyl 4,5-epoxy-3-methylcyclohexane-1,2-dicarboxylate; benzyl isononyl 4,5-epoxy-3-methylcyclohexane-1,2-dicarboxylate; benzyl 2-propylheptyl 4,5-epoxy-3-methylcyclohexane-1,2-dicarboxylate; benzyl butyl 4,5-epoxy-4-methylcyclohexane-1,2-dicarboxylate; benzyl isononyl 4,5-epoxy-4-methylcyclohexane-1,2-dicarboxylate; benzyl 2-propylheptyl 4,5-epoxy-4-methylcyclohexane-1,2-dicarboxylate; benzyl butyl 5,6-epoxynorbornane-2,3-dicarboxylate; benzyl isononyl 5,6-epoxynorbornane-2,3-dicarboxylate; benzyl 2-propylheptyl 5,6-epoxynorbornane-2,3-dicarboxylate; benzyl butyl 1-methyl-5,6-epoxynorbornane-2,3-dicarboxylate; benzyl isononyl 1-methyl-5,6-epoxynorbornane-2,3-dicarboxylate; benzyl 2-propylheptyl 1-methyl-5,6-epoxynorbornane-2,3-dicarboxylate; benzyl butyl 3,6-4,5-diepoxycyclohexane-1,2-dicarboxylate; benzyl isononyl 3,6-4,5-diepoxy-cyclohexane-1,2-dicarboxylate; benzyl 2-propylheptyl 3,6-4,5-diepoxy-cyclohexane-1,2-dicarboxylate; benzyl butyl 5,6-epoxybicyclo[2.2.2]octane-2,3-dicarboxylate; benzyl isononyl 5,6-epoxybicyclo[2.2.2]octane-2,3-dicarboxylate; benzyl 2-propylheptyl 5,6-epoxybicyclo[2.2.2]octane-2,3-dicarboxylate; butyl 1-phenylethyl 4,5-epoxycyclohexane-1,2-dicarboxylate; butyl 1-phenylethyl 4,5-epoxy-3-methylcyclohexane-1,2-dicarboxylate; isononyl 1-phenylethyl 4,5-epoxycyclohexane-1,2-dicarboxylate; isononyl 1-phenylethyl 4,5-epoxy-3-methylcyclohexane-1,2-dicarboxylate; 1-phenylethyl 2-propylheptyl 4,5-epoxycyclohexane-1,2-dicarboxylate; 1-phenylethyl 2-propylheptyl 3-methylcyclohex-4-ene-1,2-dicarboxylate; and combinations thereof.

Catalyst. A catalyst is advantageously employed in the esterification reaction(s) of the invention. The catalyst is typically a base, preferably an organic base. Suitable organic bases include pyridines, tertiary amines, room temperature ionic liquids, and combinations thereof. Tertiary amines are preferred. Tertiary amines for use in the process of the invention can be represented by the structure R²R³R⁴N where R²-R⁴ may be the same or different alkyl radicals. Examples of suitable trialkyl amines include trimethylamine, triethylamine, tripropylamine, tributylamine, tripentylamine, trihexylamine, triheptylamine, trioctylamine, trinonylamine, as well as the normal-, iso-, and tert-configurations of the foregoing, if appropriate. Various combinations of R²-R⁴ where R²-R⁴ can be individually selected from methyl, ethyl, propyl, butyl, hexyl, heptyl, octyl, nonyl, decyl are also possible. Other amines suitable herein include triisoamylamine, methyldiethylamine, dimethylethylamine, dimethylcyclohexylamine, dimethylhexylamine, diethylhexylamine, dimethyldecylamine and others. The preferred amine is triethylamine.

Pyridines are six-membered heterocycles having 5 carbon atoms and one nitrogen atom in the ring. Pyridine itself has the formula C₅H₅N, and can be formed by the following reaction:

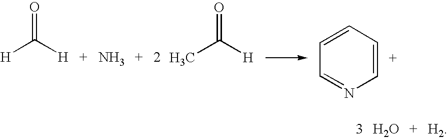

By substituting other aldehydes for acetaldehyde, one obtains alkyl and aryl substituted pyridines.

An ionic liquid is a liquid that contains essentially only ions. Some ionic liquids, such as ethylammonium nitrate are in a dynamic equilibrium where at any time more than 99.99% of the liquid is made up of ionic rather than molecular species. Broadly the term includes molten salts, for instance, sodium chloride at temperatures higher than 800° C. Salts that are liquid at room temperature are called room-temperature ionic liquids, or RTILs.

Another embodiment of the invention is a process of making an asymmetric cyclic ester comprising: (a) contacting (i) at least one cyclic dicarboxylic acid anhydride with (ii) at least one C₂-C₁₈ aliphatic alcohol, in the presence of (iii) a base to form a reaction mixture and (b) contacting the reaction mixture with a benzyl halide or phenyl-substituted alkyl halide to form a product. An illustrative example of this process is depicted in Reaction 3, where cyclohexane-1,2-dicarboxylic anhydride, triethylamine and benzyl chloride are used specifically.

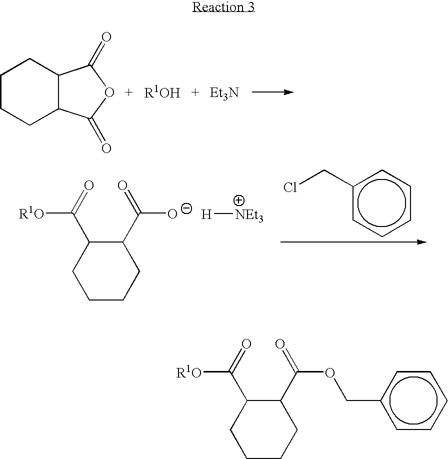

The process may further comprise, after (a)(iii), step (a)(iv), wherein step (a)(iv) includes maintaining the reaction mixture temperature at about 60 to about 130° C. The process may further comprise, after (b), step (b)(i), wherein (b)(i) includes maintaining the reaction mixture temperature at about 100 to about 180° C. The process may further comprise, after (b), step (c), wherein step (c) comprises washing the product of (b) with water at a pH of less than 6, followed by (d), washing the product of (c) with water at a pH of greater than 8, followed by (e), wherein step (e) comprises washing the product of (d) with water. The process may further comprise, after (e), step (f), wherein step (f) comprises steam stripping the product at a pressure of less than 500 torr, preferably less than 400 torr. The process may yet further include, after (f), step (g), wherein step (g) comprises stripping the product of moisture at a pressure of less than 200 torr, preferably less than 100 torr.

In a preferred embodiment of the process, the at least one cyclic carboxylic acid anhydride is selected from the group consisting of 4-cyclohexene-1,2-dicarboxylic acid anhydride; cyclohexane-1,2-dicarboxylic acid anhydride; methylcyclohexane-1,2-dicarboxylic acid anhydride; 1,2-dicarboxy-3-alkylcyclohex-3-ene anhydride; cis-5-norbornene-2,3-dicarboxylic anhydride; norbornane-2,3-dicarboxylic anhydride; methyl-5-norbornene-2,3-dicarboxylic anhydride; 3,6-epoxy-cyclohexene-1,2-dicarboxylic acid anhydride; 3,6-epoxycyclohexane-1,2-dicarboxylic acid anhydride; bicyclo[2.2.2]oct-5-ene-2,3-dicarboxylic anhydride; bicyclo[2.2.2]octane-2,3-dicarboxylic anhydride; and combinations thereof.

In another preferred embodiment of the process, the at least one cyclic carboxylic acid anhydride is selected from the group consisting of 4-cyclohexene-1,2-dicarboxylic acid anhydride and cyclohexane-1,2-dicarboxylic acid anhydride and combinations thereof, and the aliphatic alcohol is selected from the group consisting of isononyl alcohol and 2-propylheptyl alcohol, and combinations thereof. The process may utilize any base disclosed elsewhere herein, in any combination.

Another embodiment of the invention is a process of making an epoxidized asymmetric cyclic ester comprising: (a) contacting (i) a cyclic dicarboxylic anhydride including one or two unsaturated bonds with (ii) an aliphatic alcohol, in the presence of (iii) a base to form a reaction mixture, (b) contacting the reaction mixture of (a) with a benzyl halide or phenyl-substituted alkyl halide, to form a second reaction mixture, (c) isolating the resulting unsaturated diester and (d) contacting the unsaturated diester with a peracid. An illustrative example of this process is depicted in Reaction 4, where 4-cyclohexene-1,2-dicarboxylic anhydride, triethylamine and benzyl chloride are used specifically.

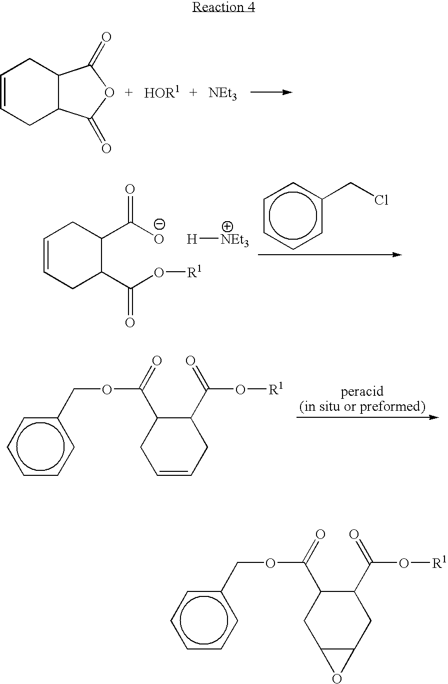

Peracids. Peracids, or peroxyacids, are excellent epoxidizing agents. For the organic peracids, there is an extra oxygen atom between the carbonyl group and their acidic hydrogen, making them electrophilic towards oxygen. Inorganic peracids include perchloric acid, HClO₄ or perbromic acid, HBrO₄. Attack at the oxygen position by a nucleophile displaces carboxylate, which is a good leaving group. An example of one such reaction involves ethylene and peroxyformic acid, or more appropriately for the invention, the reaction between benzyl isononyl cyclohex-4-ene and a peracid such as m-chloroperbenzoic acid. Other suitable peracids herein include peracetic acid (CH₃C(═O)OOH), and perbenzoic acid (C₆H₅C(═O)OOH). The peracid can be formed in situ from the addition of hydrogen peroxide to an acid, e.g. formic acid and hydrogen peroxide will form performic acid (HC(═O)OOH) within a reaction mixture. The reaction mechanism is essentially an electrophilic attack, with a proton being transferred from the epoxide oxygen to the carboxylic acid by-product. First, the nucleophilic π (pi-) bond donates its electrons to the oxygen, breaking the O—O bond to form the new carbonyl bond. The electrons from the old O—H bond make up the second new C—O bond, and the original carbonyl group uses its electrons to pick up the proton.

Still another embodiment of the invention is a process of making a saturated asymmetric cyclic ester comprising (a) contacting (i) at least one cyclic dicarboxylic anhydride including one or two unsaturated bonds with (ii) an aliphatic alcohol, in the presence of (iii) a base to form a reaction mixture, (b) contacting the reaction mixture of (a) with a benzyl halide or phenyl-substituted alkyl halide, to form a second reaction mixture, (c) isolating the resulting unsaturated diester, and (d) contacting the unsaturated diester with a hydrogenation catalyst and hydrogen gas. An illustrative example of this process is depicted in Reaction 5, where 4-cyclohexene-1,2-dicarboxylic anhydride, triethylamine and benzyl chloride are used specifically.

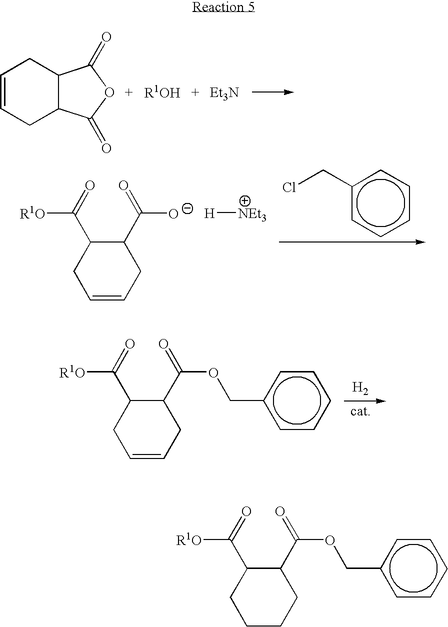

Yet another embodiment of the invention is a process of making a saturated asymmetric cyclic ester comprising: (a) contacting (i) at least one cyclic dicarboxylic anhydride with (ii) benzyl alcohol or phenyl-substituted alcohol, in the presence of a base to form a reaction mixture, and (b) contacting the reaction mixture of (a) with at least one C₂-C₁₈ alkyl halide to form said mixed diester. An illustrative example of this process is depicted in Reaction 6, where cyclohexane-1,2-dicarboxylic anhydride, triethylamine and benzyl alcohol are used specifically.

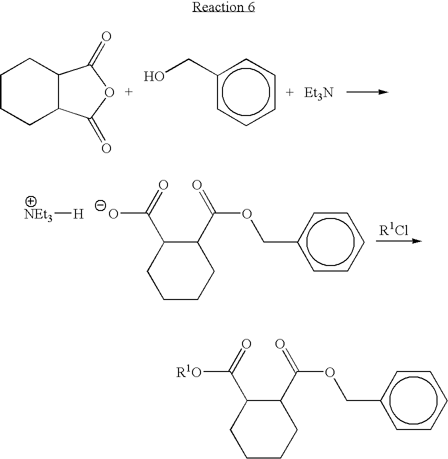

Still another embodiment of the invention is a process of making an asymmetric cyclic ester comprising: (a) contacting (i) a maleic anhydride with (ii) an aliphatic alcohol, in the presence of (iii) a base to form a reaction mixture, (b) contacting the reaction mixture of (a) with a benzyl halide or phenyl-substituted alkyl halide, to form an asymmetric maleate diester, and (c) contacting the ester of (b) with a diene to form an asymmetric cyclic ester. In a preferred embodiment, the diene is selected from the group consisting of butadiene; 3-sulfolene; isoprene; 1,3-pentadiene; cyclopentadiene; furan; 1-methoxybutadiene; 1,3-hexadiene; 3-methyl-1,3-pentadiene; 4-methyl-1,3-pentadiene; 1,3-cyclohexadiene; sorbic acid esters; ethyl sorbate; 1,2,3,4,5-pentamethylcyclopentadiene; myrcene (7-methyl-3-methylene-1,6-octadiene); and combinations thereof. An illustrative example of this process is depicted in Reaction 7, where maleic anhydride, triethylamine, benzyl chloride, and 1,3-butadiene are used specifically.

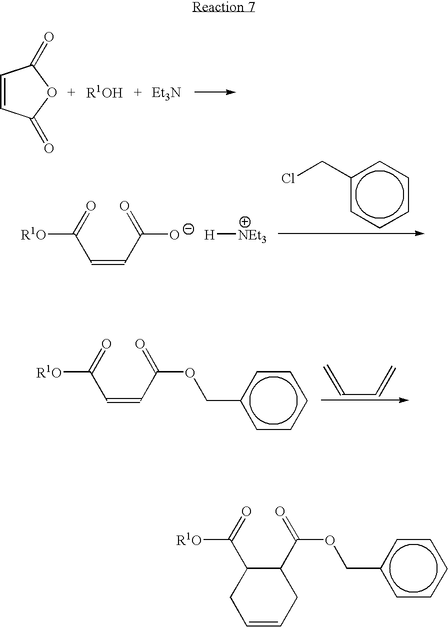

This process may further comprise contacting the unsaturated asymmetric cyclic ester product with a hydrogenation catalyst and hydrogen gas. This process may yet further include contacting the unsaturated asymmetric cyclic ester product with a peracid.

An embodiment of the invention is a process of making a saturated asymmetric cyclic ester comprising: (a) contacting (i) a cyclic dicarboxylic anhydride including at least one unsaturated bond with (ii) an aliphatic alcohol, in the presence of (iii) a base to form a reaction mixture including an asymmetric monoester salt, (b) contacting with hydrogen (i) an unsaturated asymmetric monoester acid salt formed in (a) in the presence of (ii) a hydrogenation catalyst, to form a reaction mixture, and (c) contacting the reaction mixture of (b) with a benzyl halide or phenyl-substituted alkyl halide, to form an asymmetric cyclic ester. In a preferred embodiment of this process, the cyclic dicarboxylic anhydride is phthalic anhydride. Illustrative examples of this process are depicted in Reaction 8 and Reaction 9.

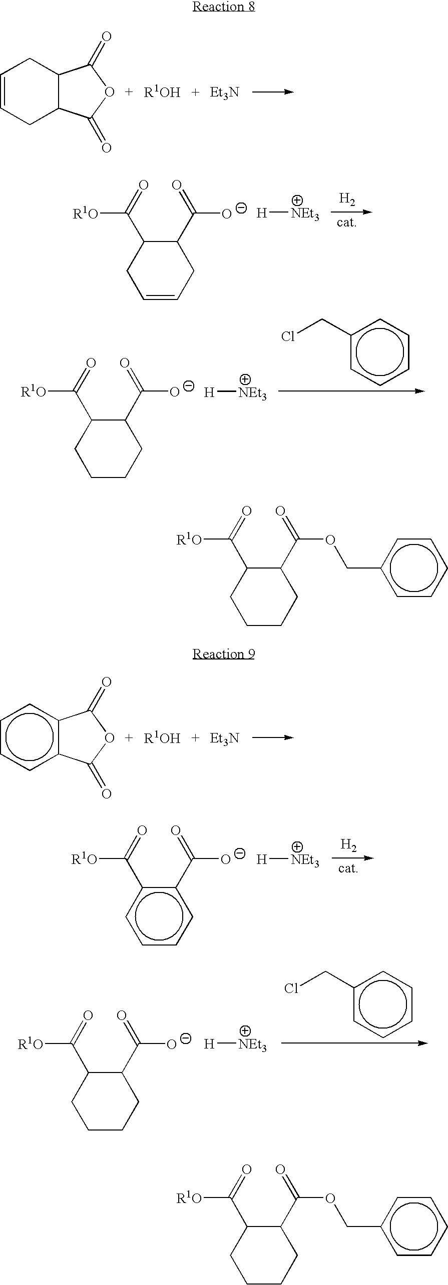

Another embodiment of the invention is a process of making an asymmetric cyclic ester comprising: (a) contacting (i) a maleic anhydride with (ii) a benzyl alcohol or a phenyl-substituted alcohol, in the presence of (iii) a base to form a reaction mixture, (b) contacting the reaction mixture of (a) with an alkyl halide to form an asymmetric maleate diester, and (c) contacting the ester of (b) with a diene to form an asymmetric cyclic ester. In a preferred embodiment of the process, the diene may be selected from the group consisting of butadiene; 3-sulfolene; isoprene; 1,3-pentadiene; cyclopentadiene; furan; 1-methoxybutadiene; 1,3-hexadiene; 3-methyl-1,3-pentadiene; 4-methyl-1,3-pentadiene; 1,3-cyclohexadiene; sorbic acid esters; ethyl sorbate; myrcene (7-methyl-3-methylene-1,6-octadiene); 1,2,3,4,5-pentamethylcyclopentadiene; and combinations thereof. An illustrative example of this process is depicted in Reaction 10.

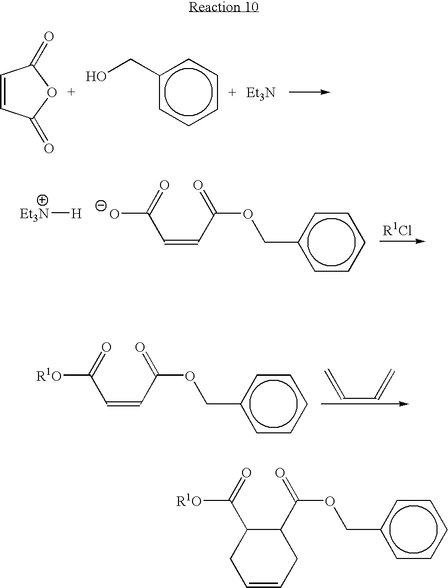

Another embodiment of the invention is a process of making a 1,2-cyclic alkyl/aryl mixed diester comprising (a) contacting (i) at least one cyclic dicarboxylic acid anhydride with (ii) a C₂-C₁₈ alkyl alcohol, in the presence of (iii) a trialkyl amine to form a reaction mixture and (b) contacting the reaction mixture of (a) with a benzyl halide or phenyl-substituted alkyl halide, to form a product. The reaction mixture may optionally be heated after the trialkyl amine is fully added to the cyclic dicarboxylic acid or anhydride and the alcohol. After full reaction, the process may further comprise washing the product with water at a pH of less than 6, followed by washing the product of with water at a pH of greater than 8. The product may also be washed with plain water such as tap water, distilled water or deionized water. After washing, the product may be steam stripped at a pressure of less than 500 torr, and also the moisture may be removed by stripping at a pressure of less than 200 torr.

In a preferred embodiment of the process, the cyclic compound is 1,2-dicarboxy cyclohex-4-ene anhydride and the aliphatic alcohol is isononyl alcohol.

Still another embodiment of the invention is a process of making an epoxidized asymmetric cyclic ester comprising: (a) contacting (i) a cyclic dicarboxylic anhydride including one or two unsaturated bonds with (ii) an aliphatic alcohol, in the presence of (iii) a base to form a reaction mixture, (b) contacting the reaction mixture of (a) with a benzyl halide or phenyl-substituted alkyl halide, to form a second reaction mixture, (c) isolating the resulting unsaturated diester and (d) contacting the unsaturated diester with a peracid.

A further embodiment of the invention is a process of making a saturated 1,2-cyclic alkyl/aryl mixed diester comprising: (a) contacting (i) at least one cyclic dicarboxylic anhydride with (ii) an alkyl alcohol, in the presence of (iii) a trialkyl amine to form a reaction mixture, (b) contacting the reaction mixture of (a) with a benzyl halide or phenyl-substituted alkyl halide, to form a second reaction mixture, (c) isolating the resulting saturated diester, and (d) contacting the saturated diester with a hydrogenation catalyst and hydrogen gas.

Yet another embodiment of the invention is a process of making a saturated 1,2-cyclic alkyl/aryl mixed diester comprising: (a) contacting (i) at least one cyclic dicarboxylic anhydride with (ii) benzyl alcohol or phenyl-substituted alcohol, to form a benzyl half-ester, and (b) contacting the benzyl half ester with (i) a trialkyl amine and then (ii) a C₂-C₁₈ alkyl halide to form said mixed diester.

The asymmetric cyclic esters of this invention are predominantly of the cis-configuration. Isomerization of the predominant cis-form to a predominant trans-form can be effected using methods such as disclosed in U.S. Pat. No. 5,231,218.

Another embodiment of the invention is a process of plasticizing a polymer comprising contacting any compound disclosed herein with a polymer.

The asymmetric cyclic esters of this invention can be combined with other known plasticizers to formulate a plasticizer package to be used in making plasticized thermoplastic polymers. Examples of known plasticizers that might be used in conjunction with a plasticizer of the invention include, without limitation, the following.

(a) Phosphate plasticizers such as triphenyl phosphate, tricresyl phosphate, trioctyl phosphate, octyl diphenyl phosphate, trichlorethyl phosphate, cresyl diphenyl phosphate, tributyl phosphate, tri-2-ethylhexyl phosphate and triphenyl phosphate.

(b) Phthalate ester plasticizers such as dimethyl phthalate, diethyl phthalate, diisobutyl phthalate, dibutyl phthalate, diheptyl phthalate, di-2-ethyl hexyl phthalate, diisooctyl phthalate, di-n-octyl phthalate, dinonyl phthalate, diisononyl phthalate, diisodecyl phthalate, diundecyl phthalate, ditridecyl phthalate, dicyclohexyl phthalate, butyl benzyl phthalate, benzyl isononyl phthalate, butyl lauryl phthalate, methyl octyl phthalate, and octyl decyl phthalate.

(c) Aromatic carboxylic acid ester plasticizers such as trioctyl trimellitate, tri-n-octyl trimellitate, triisooctyl trimellitate, dioctyl terephthalate, and octyl oxybenzoate.

(d) Aliphatic dibasic acid ester plasticizers such as dibutyl adipate, di-n-hexyl adipate, di-2-ethylhexyl adipate, di-n-octyl adipate, n-octyl-n-decyl adipate, diisononyl adipate, diisodecyl adipate, dicapryl adipate, di-2-ethylhexyl azelate, dimethyl sebacate, diethyl sebacate, dibutyl sebacate, di-n-octyl sebacate, di-2-ethylhexyl sebacate, di-2-ethoxyethyl sebacate, dioctyl succinate, diisodecyl succinate, dioctyl tetrahydrophthalate, di-n-octyl tetrahydrophthalate, di-2-ethylhexyl cyclohexane-1,2-dicarboxylate, and diisononyl cyclohexane-1 2-dicarboxylate.

(e) Fatty acid ester derivatives such as butyl oleate, acetyl methyl ricinoleate, pentaerythritol ester, dipentaerythritol hexaester, triacetin and tributylene.

(f) Oxyacid ester plasticizers such as acetyl methyl ricinoleate, acetyl butyl ricinoleate, butyl phthalyl butyl glycolate and acetyl tributyl citrate.

(g) Epoxy plasticizers such as epoxidized soybean oil, epoxidized flaxseed oil, epoxy butyl stearate, epoxy decyl stearate, epoxy octyl stearate, epoxy benzyl stearate, epoxy dioctyl hexahydrophthalate and epoxy didecyl hexahydrophthalate.

(h) Dihydric alcohol ester plasticizers such as diethylene glycol dibenzoate, dipropylene glycol dibenzoate, and triethylene glycol di-2-ethyl butyrate.

(i) Chlorine-containing plasticizers such as chlorinated paraffin, chlorinated diphenyl, chlorinated methyl fatty acids and methoxychlorinated methyl fatty acids.

(j) Polyester plasticizers such as polypropylene adipate, polypropylene sebacate, polyester and acetylated polyester.

(k) Sulfonic acid derivatives such as p-toluenesulfonamide, o-toluenesulfonamide, p-toluene sulfone ethylamide, o-toluene sulfone ethyl amide, toluene sulfone-N-ethylamide and p-toluene sulfone-N-cyclohexylamide.

(l) Citric acid derivatives such as triethyl citrate, acetyl triethyl citrate, tributyl citrate, acetyl tributyl citrate, acetyl tri-2-ethylhexyl citrate and acetyl noctyldecyl citrate.

(m) Other plasticizers not falling into one of the above categories, such as alkyl pyrrolidones surfactants, alkyl imidazoles, N-alkyl hexahydrophthalimide, dibutyl fumarate, dioctyl fumarate, linear alkyl benzenes, and mineral oil.

The following experimental examples serve to illustrate, and not limit, the scope of the invention. Example 1. A one-liter four-necked round bottom flask was charged with 152 g of 4-cyclohexene-1,2-dicarboxylic acid anhydride and 152 g of isononyl alcohol. A 250 ml addition funnel was charged with 107 g of triethylamine, and a 125 ml addition funnel was charged with 134 g of benzyl chloride. The reaction was blanketed with nitrogen. The reaction was agitated at 250 RPM, and one third of the triethylamine was added to the reaction. The reaction was heated to 125° C. and was stirred at 125° C. for 5 minutes. The reaction was then cooled to 100° C., and the rest of the triethylamine was added to the reaction. The reaction was stirred at 100-105° C. for 5 minutes.

The agitation was increased to 300 RPM and the reaction was heated to 120° C. The addition of the benzyl chloride was started dropwise. The reaction temperature rose upon the addition of the benzyl chloride. The temperature was then controlled at 145° C. The addition of the benzyl chloride was completed over 30 minutes. The reaction was then stirred for an additional 90 minutes after the addition of the benzyl chloride was completed.

The product was washed first with 100 g of water at a pH of 2 and then with 100 g of water at a pH of 12. The product was then washed with 100 g of water. The material was steam stripped at 125° C. at 80 mm Hg pressure. The product was then stripped of water at 125° C. at 50 mm Hg to give 328.1 g (85%) of benzyl isononyl 4-cyclohexene-1,2-dicarboxylate.

Example 2. Using the procedure of Example 1, 145 g of 1,2-cyclohexanedicarboxylic anhydride were reacted with 144 g of isononyl alcohol and 128 g of benzyl chloride to give 338 g (96.5%) of benzyl isononyl cyclohexane-1,2-dicarboxylate.

Example 3. Using the procedure of in Example 1, 153 g of 4-cyclohexene-1,2-dicarboxylic acid anhydride were reacted with 78 g of butanol and 134 g of benzyl chloride to yield 283 g (95.1%) of benzyl butyl 4-cyclohexene-1,2-cbuoxyilate.

Example 4. Using the procedure of Example 1, 103 g of 1,2-cyclohexanedicarboxylic anhydride were reacted with 52 g of butanol and 90 g of benzyl chloride to yield 191 g (90.1%) of benzyl butyl cyclohexane-1,2-dicarboxylate.

Example 5. Using the procedure of Example 1, 152 g of 4-cyclohexene-1,2-dicarboxylic acid anhydride were reacted with 158 g of 2-propylheptyl alcohol and 136 g of benzyl chloride to yield 356 g (88.9%) of benzyl 2-propylheptyl 4-cyclohexene-1,2-dicarboxylate.

Example 6. Using the procedure of Example 1, 153g of 1,2-cyclohexanedicarboxylic anhydride were reacted with 166 g of 2-propylheptyl alcohol and 134 g of benzyl chloride to yield 382 g (89.9%) of benzyl 2-propylheptyl cylcohexane-1,2-dicarboxylate.

Example 7. A solution of 47.5 g of m-chloroperbenzoic acid in 511 g of chloroform was placed in a 1 L round bottom flask. The solution temperature was kept between 25-35° C. by means of a water bath while 99.88 g of the product of Example 1 was added dropwise to the chloroform solution over the course of 2 hours. The reaction was stirred at 25° C. overnight, i.e., ˜12 hours. The next day the reaction solution was filtered and was extracted with 100 g of 10% sodium bicarbonate solution. The chloroform was then removed under vacuum to give 100.55 g of the epoxidized product, benzyl isononyl 4,5-epoxycyclohexane-1,2-dicarboxylate.

Example 8. A Parr stirred pressure reactor was charged with 97.44 g of the product of Example 5 and 0.98 g of 5% Pt on carbon. The reactor was sealed and purged with nitrogen. The reactor was then heated to 42° C. and was pressurized to 100 psig with hydrogen. The reactor was stirred for 4.5 hours while maintaining the temperature at 42° C. and hydrogen pressure at 100 psig. The reactor was cooled to room temperature and the excess pressure was released. The reaction product was filtered through Celite to give 73.9 g (75%) of product. GC analysis of the product indicated that 89.8% of the product benzyl 2-propylheptyl cyclohexane-1,2-dicarboxylate.

Example 9. A 1 L four-necked round bottom flask was charged with 152.76 g of 4-cyclohexene-1,2-dicarboxylic anhydride and 153.77 g of isononyl alcohol. A 250 ml addition funnel was charged with 106.77 g of triethylamine. The reaction was blanketed with nitrogen. The reaction was agitated at 250 RPM and one-third of the triethylamine was added to the reaction. The reaction quickly rose to 125° C. The reaction was stirred at 125±2° C. for 5 minutes. The reaction was then cooled to 100° C., and the rest of the triethylamine was added to the reaction. The reaction was stirred at 100-105° C. for 5 minutes before it was cooled to room temperature to give 407.34 g (98.6%) of monoisononyl ester of 4-cyclohexene-1,2-dicarboxylic acid triethylamine salt.

Example 10. A Parr stirred pressure reactor was charged with 98.92 g of the product of Example 9 and 1.00 g of 5% Pt on carbon. The reactor was sealed and purged with nitrogen. The reactor was then heated to 38° C. and was pressurized to 100 psig with hydrogen. The reactor was stirred for 4.5 hours while maintaining the temperature at 42° C. and hydrogen pressure at 100 psig. The reactor was cooled to room temperature and the excess pressure was released. The reaction product was filtered through Celite to give 96.55 g (96.9%) of product. GC analysis of the product indicated that 91.3% of the product was the monoisononyl ester of cyclohexane-1,2-dicarboxylic acid triethylamine salt.

Example 11. A 1 L reactor was charged with 270.50 g of material prepared as described in Example 10. The agitation was set at 300 RPM and the reaction was heated to 120° C. The addition of the benzyl chloride was started drop wise. The reaction temperature rose upon the addition of the benzyl chloride, and the temperature was then controlled at 145° C. The addition of the benzyl chloride was completed over 30 minutes. After the addition was complete, the reaction was stirred for an additional 90 minutes. The reaction was cooled to 100° C., and the product was washed first with 100 g of water at a pH of 2 and then with 100 g of water at a pH of 12. The product was then washed with 100 g of water. The material was steam stripped at 125° C. at 80 mm Hg pressure. The product was then stripped of water at 125° at 50 mm Hg to give 126.72 g (50.5%) of benzyl isononyl cyclohexane-1,2-dicarboxylate.

Example 12. A 1 L four-necked round bottom flask was charged with 166.3 g of 4-methyl-4,5-cyclohexene-1,2-dicarboxylic anhydride and 152.18 g of isononyl alcohol. A 250 ml addition funnel was charged with 107 g of triethylamine, and a 125 ml addition funnel was charged with 134 g of benzyl chloride. The reaction was blanketed with nitrogen. The reaction was agitated at 250 RPM, and one third of the triethylamine was added to the reaction. The reaction was heated to 125° C. and was stirred at 125° C. for 5 minutes. The reaction was then cooled to 100° C., and the rest of the triethylamine was added to the reaction. The reaction was stirred at 100-105° C. for 5 minutes. The agitation was increased to 300 RPM and the reaction was heated to 120° C. The addition of the benzyl chloride was started drop wise. The reaction temperature rose upon the addition of the benzyl chloride. The temperature was then controlled at 145° C. The addition of the benzyl chloride was completed over 30 minutes. The reaction was then stirred for an additional 90 minutes after the addition of the benzyl chloride was completed. The product was washed first with 100 g of water at a pH of 2 and then with 100 g of water at a pH of 12. The product was then washed with 100 g of water. The material was steam stripped at 125° C. at 80 mm Hg pressure. The product was then stripped of water at 125° C. at 50 mm Hg to give 328.1 g (85%) of benzyl isononyl 4-methyl-4,5-cyclohexene-1,2-dicarboxylate.

Example 13. A 500 ml four-necked round bottom flask, fitted with a mechanical agitator, was charged with 104.24 g of 1,2-cyclohexanedicarboxylic anhydride and 88.95 g of 2-ethylhexanol. To this assembly were added a thermocouple and Friedrichs condenser with nitrogen inlet. A 250 ml addition funnel charged with 69.77 g of triethylamine, and another 250 ml addition funnel charged with 96.05 g of 1-chloro-1-phenylethane (93.6% purity by GC) were connected via a Claisen adapter to the assembly. The reaction was blanketed with nitrogen. The reaction was stirred and one-third of the triethylamine was added to the reaction. The reaction quickly rose to 120° C. The reaction was then cooled to 99° C. over 10 minutes. Then the remainder of the triethylamine was added to the reaction. The reaction was stirred at 90° C. for 10 minutes. Addition of 1-chloro-1-phenylethane took 8 minutes after which the reaction temperature was 89° C. With heating the reaction temperature rose to 142° C. over 38 minutes whereupon salts were visible in the reaction mixture. Reaction was maintained at 142° C. for 2 hours before it was cooled to 110° C. Water (100 ml) was added and the mixture acidified to a pH of <4.5. The organic layer was washed with 200 ml water. The organic was then treated with 150 ml water and 50% NaOH (11.6 g) to give a mixture with a pH of >9. The organic layer was again washed with 200 ml water. The organic portion was steam stripped at 123-4° C. at 50 mm Hg pressure. Water was distilled from the product at up to 132° C. at 50 mm Hg to give 183.1 g (70%) of 2-ethylhexyl 1-phenylethyl cyclohexane-1,2-dicarboxylate that was of 93.3% purity by GC.

Comparative Testing: Benzyl isononyl 4-cyclohexene-1,2-dicarboxylate (Compound A) and benzyl isononyl cyclohexane-1,2-dicarboxylate (Compound B) are useful in some of the same applications as butyl benzyl phthalate (BBP). The inventive compounds were compared to three known plasticizer products: BBP, a fast fusing phthalate, commercially available from Ferro Corporation, Cleveland, Ohio, as Santicizer® 160; di-isononyl phthalate (DINP), a widely used phthalate; and diisononyl cyclohexane-1,2-dicarboxylate (DINCH), commercially available from BASF Corporation of Florham Park, N.J., as Hexamoll® DINCH.

The parameters presented in Table 1 are as follows: Volatility—Activated Carbon Method—1 DAY (Ref: ASTM 1203); Volatility—Activated Carbon Method—6 DAY (Ref: ASTM 1203) Kerosene Extraction (Ref: ASTM D1239, D543); Brabender Fusion times: ASTM D2538-02 Standard Practice for Fusion of Poly(Vinyl Chloride) (PVC) Compounds Using a Torque Rheometer. Cold Flex Temp: (Ref: ASTM D1043-02 Standard Test Method for Stiffness Properties of Plastics as a Function of Temperature by Means of a Torsion Test (Clash-Berg Cold Flex Temperature Method); Shore A Hardness (Ref: ASTM D2240-05 Standard Test Method for Rubber Property-Durometer Hardness (Shore A Hardness) and ASTM D618-05 Standard Practice for Conditioning Plastics for Testing). Water sensitivity testing, ASTM D1239-07: Standard Test Method for Resistance of Plastic Films to Extraction by Chemicals, is a rapid test to determine plasticizer loss from plastic film when immersed in liquids commonly used in households.

The inventive examples are COMPOUND A, in Inventive Example (Ex1) and COMPOUND B, in Inventive Example 2 (Ex2), while the comparative (prior art) examples are CE1, CE2, and CE3.

The inventive products COMPOUND A and COMPOUND B outperformed DINCH in all tests except cold flex temperature. The products performed between BBP and DINP in all the tests; outperforming DINP with faster fusion time, softer Shore A, and far lower kerosene extraction, and outperforming BBP in cold flex and having lower volatility.

TABLE 1
Comparative Property Testing Data Summary.

| | EX1 | EX2 | CE1 | CE2 | CE3 |
|---|---|---|---|---|---|
| | Compound A | Compound B | BBP | DINP | DINCH |
| Cold Flex | −25 | −21.8 | −17.2 | −31.8 | −41.8 |
| Temp. | | | | | |
| (degrees C.) | | | | | |
| Kerosene Ext. | 10.84 | 11.98 | 3.88 | 70.22 | 72.6 |
| (%) | | | | | |
| Hardness | 68.7 | 70.2 | 68.5 | 71 | 71.8 |
| (Shore A) | | | | | |
| Volatility 1 day | 3.12 | 3.09 | 8.82 | 1.53 | 3.21 |
| (%) | | | | | |
| Volatility 6 day | 15.48 | 15.71 | 42.26 | 8.73 | 16.15 |
| (%) | | | | | |
| H₂O Sensitivity | −0.04 | −0.03 | −0.1 | 0.03 | −0.03 |
| (%) | | | | | |
| Fusion Time | 1:56 | 1:46 | 1:12 | 2:01 | 5:28 |
| (min) | | | | | |

It will be appreciated that although the examples herein primarily concern a plasticizer for use in PVC resins, the use of the compositions disclosed herein is also envisioned with a variety of thermoplastic polymer resins, elastomers, and thermoplastic elastomer compositions.

Additional advantages and modifications will readily occur to those skilled in the art. Therefore, the invention in its broader aspects is not limited to the specific details and illustrative example shown and described herein. Accordingly, various modifications may be made without departing from the spirit or scope of the general invention concept as defined by the appended claims and their equivalents.

1. A compound including an asymmetric cyclic ester having the formula (I): 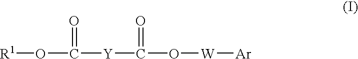
wherein Y is a C₅ to C₈ cyclic alkyl group,
wherein R¹ is a straight chain or branched C₂-C₁₈ alkyl group,
wherein W is a straight chain or branched C₁-C₅ alkyl group,
wherein Ar is a C₆-C₁₅ cyclic aryl group having at least three double bonds, and
wherein R¹O(O═)C‥ and ArWO(O═)C— are attached to vicinal carbons of Y.

2. A blend of the compounds of claim 1 including at least two different types of at least one of the Y, R¹, W, and Ar groups.

3. The compound of claim 1, wherein Y is selected from the group consisting of cyclohexene, cyclohexane, and combinations thereof.

4. The compound of claim 1, wherein Y is cyclohexene oxide.

5. The compound of claim 1, wherein Y is selected from the group consisting of cyclopentadiene; cyclopentene; cyclopentane; cycloheptadiene; cycloheptene; cycloheptane; 3-methylcyclohex-4-ene; 4-methylcyclohex-4-ene; 3-methylcyclohexane; 4-methylcyclohexane; 3,6-epoxycyclohexene; 3,6-epoxycyclohexane; cis-5-norbornene; norbornene; methyl-5-norbornene; bicyclo[2.2.2]oct-5-ene; bicyclo[2.2.2]octane; and combinations thereof.

6. The compound of claim 1, wherein Y is a residue of a carboxylic acid anhydride

7. The compound of claim 6, wherein the carboxylic acid anhydride is selected from the group consisting of 4-cyclohexene-1,2-dicarboxylic acid anhydride; cyclohexane-1,2-dicarboxylic acid anhydride; 1,2-dicarboxy-4-alkyl cyclohex-4-ene anhydride; 1,2-dicarboxy-3-alkyl cyclohex-3-ene anhydride; cis-5-norbornene-2,3-dicarboxylic anhydride; norbornane-2,3-dicarboxylic anhydride; methyl-5-norbornene-2,3-dicarboxylic anhydride; 3,6-epoxy-cyclohexene-1,2-dicarboxylic acid anhydride; 3,6-epoxycyclohexane-1,2-dicarboxylic acid anhydride; bicyclo[2.2.2]oct-5-ene-2,3-dicarboxylic anhydride; bicyclo[2.2.2]octane-2,3-dicarboxylic anhydride; and combinations thereof.

8. The compound of claim 1, wherein R¹ is selected from the group consisting of C₄ to C₁₀ straight chain or branched alkyl groups.

9. The compound of claim 1, wherein Ar is selected from the group consisting of benzene; methylbenzene; dimethyl benzene, ditertiary butyl benzene; napththalene, anthracene, cumene and combinations thereof.

10. A process of making an asymmetric cyclic ester comprising:
a. contacting
i. at least one cyclic dicarboxylic acid anhydride with
ii. at least one C₂-C₁₈ aliphatic alcohol, in the presence of
iii. a base to form a reaction mixture and
b. contacting the reaction mixture with a benzyl halide to form a product.

11. The process of claim 10, further comprising, after (a)(iii), step (a)(iv), wherein step (a)(iv) includes maintaining the reaction mixture temperature at about 60 to about 130° C.

12. The process of claim 10, further comprising, after (b), step (b)(i), wherein (b)(i) includes maintaining the reaction mixture temperature at about 100 to about 180° C.

13. The process of claim 10, further comprising, after (b), step (c), wherein step (c) comprises washing the product of (b) with water at a pH of less than 6, followed by (d), washing the product of (c) with water at a pH of greater than 8, followed by (e), wherein step (e) comprises washing the product of (d) with water.

14. The process of claim 13, further comprising, after (e), step (f), wherein step (f) comprises steam stripping the product at a pressure of less than 500 torr.

15. The process of claim 14, further comprising after (f), step (g), wherein step (g) comprises stripping the product of moisture at a pressure of less than 200 torr.

16. The process of claim 10, wherein the at least one cyclic carboxylic acid anhydride is selected from the group consisting of 4-cyclohexene-1,2-dicarboxylic acid anhydride; cyclohexane-1,2-dicarboxylic acid anhydride; 1,2-dicarboxy-3-alkyl cyclohex-3-ene anhydride; cis-5-norbornene-2,3-dicarboxylic anhydride; norbornane-2,3-dicarboxylic anhydride; methyl-5-norbornene-2,3-dicarboxylic anhydride; 3,6-epoxy-cyclohexene-1,2-dicarboxylic acid anhydride; 3,6-epoxycyclohexane-1,2-dicarboxylic acid anhydride; bicyclo [2.2.2]oct-5-ene-2,3-dicarboxylic anhydride; bicyclo[2.2.2]octane-2,3-dicarboxylic anhydride; and combinations thereof.

17. The process of claim 10, wherein the at least one cyclic carboxylic acid anhydride of (a)(i) is selected from the group consisting of 4-cyclohexene-1,2-dicarboxylic acid anhydride and cyclohexane-1,2-dicarboxylic acid anhydride and combinations thereof, and wherein the aliphatic alcohol of (a)(ii) is selected from the group consisting of isononyl alcohol and 2-propylheptyl alcohol, and combinations thereof.

18. The process of claim 10, wherein the base is selected from the group consisting of pyridines, tertiary amines, room temperature ionic liquids, and combinations thereof.

19. A process of making an epoxidized asymmetric cyclic ester comprising:
a. contacting
i. a cyclic dicarboxylic anhydride including one or two unsaturated bonds with
ii. an aliphatic alcohol, in the presence of
iii. a base to form a reaction mixture
b. contacting the reaction mixture of (a) with a benzyl halide or a phenyl-substituted alkyl halide, to form a second reaction mixture,
c. isolating the resulting unsaturated diester and
d. contacting the unsaturated diester with a peracid.

20. A process of making a saturated asymmetric cyclic ester comprising:
a. contacting
i. at least one cyclic dicarboxylic anhydride including one or two unsaturated bonds with
ii. an aliphatic alcohol, in the presence of
iii. a base to form a reaction mixture,
b. contacting the reaction mixture of (a) with a benzyl halide or a phenyl-substituted alkyl halide, to form a second reaction mixture,
c. isolating the resulting unsaturated diester, and
d. contacting the unsaturated diester with a hydrogenation catalyst and hydrogen gas.

21. A process of making a saturated asymmetric cyclic ester comprising:
a. contacting
i. at least one cyclic dicarboxylic anhydride with
ii. benzyl alcohol or phenyl-substituted alcohol, in the presence of a base to form a reaction mixture, and
b. contacting the reaction mixture of (a) with at least one C₂-C₁₈ alkyl halide to form said mixed diester.

22. A process of making an asymmetric cyclic ester comprising:
a. contacting
i. a maleic anhydride with
ii. an aliphatic alcohol, in the presence of
iii. a base to form a reaction mixture,
b. contacting the reaction mixture of (a) with a benzyl halide or a phenyl-substituted alkyl halide, to form an asymmetric maleate diester, and
c. contacting the ester of (b) with a diene to form an asymmetric cyclic ester.

23. The process of claim 22, wherein the diene is selected from the group consisting of butadiene; 3-sulfolene; isoprene; 1,3-pentadiene; cyclopentadiene; furan; 1-methoxybutadiene; 1,3-hexadiene; 3-methyl-1,3-pentadiene; 4-methyl-1,3-pentadiene; 1,3-cyclohexadiene; sorbic acid esters; ethyl sorbate; 1,2,3,4,5-pentamethylcyclopentadiene; 7-methyl-3-methylene-1,6-octadiene; and combinations thereof.

24. The process of claim 22, further comprising contacting the product with a hydrogenation catalyst and hydrogen gas.

25. The process of claim 22, further comprising contacting the product with a peracid.

26. A process of making a saturated asymmetric cyclic ester comprising:
a. contacting
i. a cyclic dicarboxylic anhydride including one, two or three unsaturated bonds with
ii. an aliphatic alcohol, in the presence of
iii. a base to form a reaction mixture including an asymmetric monoester salt,
b. hydrogenating
i. an asymmetric monoester acid salt formed in (a) in the presence of
ii. a hydrogenation catalyst, to form a reaction mixture, and
c. contacting the reaction mixture of (b) with a benzyl halide or a phenyl-substituted alkyl halide, to form an asymmetric cyclic ester.

27. The process of claim 26, wherein the cyclic dicarboxylic anhydride is phthalic anhydride.

28. A process of making an asymmetric cyclic ester comprising:
a. contacting
i. a maleic anhydride with
ii. a benzyl alcohol or a phenyl-substituted alcohol, in the presence of
iii. a base to form a reaction mixture,
b. contacting the reaction mixture of (a) with an alkyl halide to form an asymmetric maleate diester, and
c. contacting the ester of (b) with a diene to form an asymmetric cyclic ester.

29. The process of claim 28, wherein the diene is selected from the group consisting of butadiene; 3-sulfolene; isoprene; 1,3-pentadiene; cyclopentadiene; furan; 1-methoxybutadiene; 1,3-hexadiene; 3-methyl-1,3-pentadiene; 4-methyl-1,3-pentadiene; 1,3-cyclohexadiene; sorbic acid esters; ethyl sorbate; 1,2,3,4,5-pentamethylcyclopentadiene; 7-methyl-3-methylene-1,6-octadiene; and combinations thereof.

30. A process of plasticizing a polymer comprising contacting the plasticizer of claim 1 with a polymer.

31. A process of plasticizing a polymer comprising contacting the plasticizer of claim 2 with a polymer.
